(19) United States
(12) Patent Application Publication
(10) Pub. No.: US 2017/0354475 A1
(43) Pub. Date: Dec. 14, 2017

(54) SYSTEMS AND METHODS FOR FACILITATING CONSISTENT RADIOMETRIC TISSUE CONTACT DETECTION INDEPENDENT OF ORIENTATION

(71) Applicants: ADVANCED CARDIAC THERAPEUTICS, INC., Santa Clara, CA (US); MERIDIAN MEDICAL SYTEMS, LLC, Porland, ME (US)

(72) Inventors: Robert Chris Allison, Rancho Palos Verdes, CA (US); Jessi E. Johnson, Santa Clara, CA (US); Dorin Panescu, San Jose, CA (US); John F. McCarthy, Newbury, NH (US)

(21) Appl. No.: 15/527,984

(22) PCT Filed: Nov. 18, 2015
(86) PCT No.: PCT/US2015/061340

§ 371 Date: May 18, 2017

Related U.S. Application Data
(60) Provisional application No. 62/081,709, filed on Nov. 19, 2014.

(51) Int. Cl. A61B 90/00; A61B 18/14; A61B 5/06; A61B 18/00

(52) U.S. Cl. CPC A61B 90/06; A61B 5/068; A61B 18/1492; A61B 2018/00791; A61B 2090/065; A61B 2018/00351

(57) ABSTRACT

According to some embodiments, systems for facilitating consistent radiometric tissue contact detection independent of orientation comprise a medical instrument having an antenna positioned at a distal end, an energy delivery element positioned at a distal end of the medical instrument, a radiometer, an impedance transformation network positioned between the antenna and the radiometer, and a processor configured to receive an input signal from the radiometer and provide an output indicative of an amount of tissue contact based upon the input signal. The impedance transformation network may be tuned to provide a substantially uniform radiometric response independent of an orientation of the antenna with respect to the tissue as the antenna is brought into contact with the tissue of a subject.

### RELATED APPLICATIONS

This application claims priority to U.S. Provisional Application No. 62/081,709, filed Nov. 19, 2014, the entire content of which is incorporated herein by reference in its entirety.

### BACKGROUND

Tissue ablation may be used to treat a variety of clinical disorders. For example, tissue ablation may be used to treat cardiac arrhythmias by destroying (for example, at least partially or completely ablating, interrupting, inhibiting, terminating conduction of, otherwise affecting, etc.) aberrant pathways that would otherwise conduct abnormal electrical signals to the heart muscle. Several ablation techniques have been developed, including cryoablation, microwave ablation, radio frequency (RF) ablation, and high frequency ultrasound ablation. For cardiac applications, such techniques are typically performed by a clinician who introduces a catheter having an ablative tip to the endocardium via the venous vasculature, positions the ablative tip adjacent to what the clinician believes to be an appropriate region of the endocardium based on tactile feedback, mapping electrocardiogram (ECG) signals, anatomy, and/or fluoroscopic imaging, actuates flow of an irrigant to cool the surface of the selected region, and then actuates the ablative tip for a period of time and at a power believed sufficient to destroy tissue in the selected region.

### SUMMARY

In accordance with several embodiments, a system for facilitating determination of contact of a distal tip of a medical instrument with tissue of a subject comprises an ablation device, such as a radiofrequency ablation catheter. The ablation catheter may comprise an elongate body having a proximal end and a distal end with an antenna and an energy delivery member or element (for example, radiofrequency electrode or other ablation element) positioned at the distal end of the elongate body. The ablation catheter may comprise a radiometer positioned along the elongate body (for example, at a distal end, at a proximal end or any position between the distal end and the proximal end). The antenna and radiofrequency electrode may form a single, unitary, or integral, construct at the distal end of the elongate body. The radiofrequency electrode may be configured to contact tissue of a subject and to deliver radiofrequency energy sufficient to ablate, heat or otherwise treat the tissue. In some embodiments, the ablation catheter comprises an impedance transformation network positioned between the antenna and the radiometer configured to provide a substantially uniform or equivalent radiometric response independent of an orientation of the antenna with respect to the tissue as the antenna is brought into contact with the tissue of a subject. In some embodiments, the radiometric response is not optimized for either orientation individually but instead is designed such that the radiometric response provides sufficient contact detection accuracy in both parallel and perpendicular orientations. The impedance transformation network may comprise one or more mechanical and/or electrical components, such as capacitors, inductors and/or transmission lines.

In some embodiments, the system comprises a processor or controller (for example, a specific-purpose processor) in communication with the radiometer. The processor may be configured to, upon execution of specific instructions stored in a non-transitory computer readable medium, receive an output signal (for example, a voltage or temperature signal) from the radiometer and provide an output indicative of an amount of tissue contact based upon the output signal received from the radiometer. The system may comprise a radiofrequency generator configured to provide the radiofrequency energy to the radiofrequency electrode. In some embodiments, the generator comprises a display configured to display the output generated by the processor. The output may comprise an apparent temperature change, an indication that contact has occurred and/or an indication of a level or quality of contact. The output may provide quantitative and/or qualitative information. Contact may be determined automatically by the system or manually by an operator based upon an abrupt change in the output (for example, apparent temperature).

In some embodiments, the processor is configured to operate in a contact sensing mode or phase to determine whether the electrode is in contact with the tissue prior to delivery of ablative energy and an energy delivery mode upon determination of contact during the contact sensing mode. In one embodiment, the operational modes consist essentially of a contact sensing mode and an energy delivery mode. In some embodiments, a gain or amplification of the radiometer output signal is altered (for example, increased or decreased) during the contact sensing mode and returned to a baseline during the energy delivery mode in order to provide enhanced accuracy of contact detection. In some embodiments, the impedance transformation network is configured to provide a substantially uniform radiometric response for parallel and perpendicular electrode-tissue orientations. In some embodiments, the impedance transformation network is tuned to provide an output from the radiometer that is not substantially affected by orientation of the antenna with respect to tissue as the antenna is brought into contact with the tissue. In some embodiments, a substantially uniform radiometric response can mean that the apparent temperature change plots of temperature vs. electrode-tissue position may be substantially similar (for example, have similar slopes, amplitudes or features indicative of contact). For example, at the same electrode-tissue distance, it may be desired to have the apparent temperature change within a 20% range (for example, 0-20%, 5-10%, 10-20%, 10-15%, 5-15%, 5-20%, or overlapping ranges thereof) as the orientation of the electrode is varied from perpendicular to parallel to tissue.

The system may be used to ablate, heat or otherwise treat atrial or ventricular cardiac tissue (for example, to treat atrial fibrillation or flutter or ventricular tachycardia). In some embodiments, the processor is configured to generate an estimate of a distance between the radiofrequency electrode and the tissue. The estimate may be generated using a function, such as a mathematical model, defining an approximate relationship between the distance and a radiometric response, such as an apparent temperature. In one embodiment, the estimate is generated from a look-up table.

In one embodiment, a system for facilitating determination of contact of a distal tip of a medical instrument with tissue of a subject includes a connector (for example, a mechanical, electrical or electromechanical interface connector) configured to couple to a medical instrument having an antenna, a radiometer and an impedance transformation network positioned between the antenna and the radiometer. The embodiment of the impedance transformation network is tuned to provide a substantially uniform radiometric response independent of an orientation of the antenna with respect to the tissue as the antenna is brought into contact with the tissue of a subject (for example, regardless of whether an operator brings the distal tip of the medical tip of the instrument into contact using a parallel or perpendicular orientation). The embodiment of the system also includes a processor configured to receive an output signal from the radiometer and to generate an output indicative of an amount of tissue contact based upon the output signal from the radiometer. The processor of the embodiment of the system is configured to operate in (i) a contact sensing phase to determine whether an energy delivery element is in contact with the tissue prior to delivery of energy and (ii) an energy delivery phase upon determination of contact during the contact sensing phase. The gain of the radiometer output signal may optionally be increased during the contact sensing phase. In some embodiments, the system comprises an energy delivery module configured to generate energy to be delivered to the tissue by the medical instrument upon determination of contact. The system may include the medical instrument. In one embodiment, the medical instrument comprises an ablation device including an ablation element positioned at the distal tip of the medical instrument that is configured to deliver ablative energy to the tissue upon determination of contact.

In accordance with several embodiments, a method of facilitating a substantially uniform radiometric response that is not dependent on orientation comprises determining (for example, calculating or estimating) an impedance match between an antenna and a radiometer of an ablation catheter configured to provide a substantially equivalent radiometric response independent of orientation of the antenna with respect to tissue to be contacted and constructing an impedance transformation network based on the determined impedance match. Constructing an impedance transformation network based on the determined impedance match may comprise adjusting one or more mechanical components and/or determining values of one or more electrical filter elements (for example, LC circuits, capacitors and/or inductors, and transmission lines) of the impedance transformation network.

In some embodiments, determining an impedance match between an antenna and a radiometer of an ablation catheter configured to provide a substantially equivalent radiometric response independent of orientation of the antenna with respect to tissue to be contacted comprises evaluating radiometric responses based on contact with cardiac tissue and blood for both parallel and perpendicular orientations separately and then determining a single impedance matching value (or range of values) that will provide adequate contact detection functionality for both extreme orientations such that the contact detection functionality is sufficiently accurate without requiring the user to operate in a particular orientation. The impedance match determination may be different for different operational frequencies of the radiometer/antenna system. In some embodiments, determining the impedance match comprises determining a first impedance matching value based on a perpendicular orientation, determining a second impedance matching value based on a parallel orientation, and determining a third impedance matching value based on the first and second impedance matching values. In some embodiments, the impedance match determination is based on an amount of surface area being contacted by an energy delivery element (for example, electrode of the ablation catheter) rather than on distance measurements.

In accordance with several embodiments, a method of facilitating determination of contact between a medical instrument and tissue to be treated that is not dependent on orientation is provided. In one embodiment, the method includes determining a first impedance matching value between an antenna and a radiometer of a medical instrument to provide optimum contact sensitivity for perpendicular contact between an energy delivery member of the medical instrument and target tissue, determining a second impedance matching value between the antenna and the radiometer to provide optimum contact sensitivity for parallel contact between the energy delivery member and the target tissue, and determining a third impedance matching value based on the first and second impedance matching values to provide an adequate level of contact sensitivity whether there is perpendicular contact or parallel contact. In some embodiments, the method further includes constructing an impedance matching network to position between the antenna and the radiometer based on the third impedance matching value. Constructing the impedance matching network may include determining values of one or more capacitors, inductors or transmission lines of the impedance matching network.

In one embodiment, a medical instrument for facilitating determination of contact of a distal tip of the medical instrument with tissue of a subject comprises an antenna, a radiometer and an impedance transformation network positioned between the antenna and the radiometer to provide an output from the radiometer that is not substantially affected by orientation of the antenna with respect to tissue as the antenna is brought into contact with the tissue. The output comprises an apparent temperature change. In one embodiment, the impedance transformation network is configured to provide a substantially uniform radiometric response for parallel and perpendicular tissue contact orientations. In one embodiment, the impedance transformation network is tuned to provide the output from the radiometer that is not substantially affected by orientation of the antenna with respect to tissue as the antenna is brought into contact with the tissue.

In accordance with one embodiment, a method of determining contact between a medical instrument and tissue of a subject comprises determining whether an energy delivery member of the medical instrument is in contact with tissue based on tissue properties determined from a signal generated by a radiometer positioned at a distal end of the medical instrument. In this embodiment, the medical instrument comprises an antenna and an impedance transformation network positioned between the antenna and the radiometer. In one embodiment, the impedance transformation network is configured (for example, tuned) to provide an output from the radiometer that is not substantially affected by orientation of the antenna with respect to tissue as the antenna is brought into contact with the tissue. The step of determining whether an energy delivery member of a medical instrument is in contact with tissue may comprise generating an estimate of a distance between the energy delivery member of the medical instrument and the tissue. In one embodiment, the estimate is generated using a function, such as a mathematical model, defining an approximate relationship between the distance and a signal, such as an apparent temperature, determined from the output from the radiometer.

The methods summarized above and set forth in further detail below describe certain actions taken by a practitioner; however, it should be understood that they can also include the instruction of those actions by another party. For example, actions such as “constructing an impedance transformation network” include “instructing the constructing an impedance transformation network.” Further aspects of embodiments of the invention will be discussed in the following portions of the specification. With respect to the drawings, elements from one figure may be combined with elements from the other figures.

### BRIEF DESCRIPTION OF THE DRAWINGS

These and other features, aspects and advantages of the present application are described with reference to drawings of certain embodiments, which are intended to illustrate, but not to limit, the concepts disclosed herein. The attached drawings are provided for the purpose of illustrating concepts of at least some of the embodiments disclosed herein and may not be to scale.

FIG. 1 schematically illustrates one embodiment of an energy delivery system 10 that is configured to selectively ablate or otherwise heat or treat targeted tissue.

FIG. 2 schematically illustrates one embodiment of a distal tip of a radiofrequency ablation catheter having a microwave radiometer in contact with tissue, along with temperature and microwave zones pertaining thereto.

FIG. 3A schematically illustrates operation of a radiometer assuming no impedance mismatch.

FIG. 3B schematically illustrates results of impedance mismatch between an antenna and a radiometer of the radiofrequency ablation catheter according to one embodiment.

FIG. 4A is one embodiment of a plot illustrating that the impedance match when the antenna is in contact with tissue is better than the impedance match when the antenna is not in contact with tissue.

FIG. 4B is one embodiment of a plot illustrating apparent temperature change based on tissue/antenna separation distance.

FIGS. 5A and 5B are embodiments of plots illustrating that radiometric response typically varies significantly depending on orientation of the antenna with respect to tissue.

FIG. 6A schematically illustrates one embodiment of an impedance transformation network that may be placed between the antenna and the radiometer to affect the radiometric response.

FIG. 6B illustrates calculation of an optimum impedance match in accordance with an embodiment of the impedance transformation network of FIG. 6A.

FIG. 7 schematically illustrates one embodiment of an equivalent circuit diagram for the impedance transformation network of FIG. 6A.

FIGS. 8A and 8B are plots illustrating embodiments of the similarity in radiometric response as a result of tuning the impedance transformation network.

FIG. 9 is a graph of a contact detection function and schematically illustrates that a qualitative assessment of contact may be determined for different ranges of measurements and provided to a user.

### DETAILED DESCRIPTION

Several embodiments of the invention are particularly advantageous because they include one, several or all of the following benefits: (i) reduction in the likelihood that differences in operator use would cause significant deviation in procedural outcomes; (ii) simplification of operator instructions; (iii) optimized contact sensitivity for the operational frequency of the radiometer; (iv) impedance matching or transformation networks can be specifically designed for a particular radiometer; (v) visual feedback indicative of quality of contact that is user-friendly and easy to understand; (vi) shorter procedure times due to increased efficiency; (vii) reduction in likelihood of undesired damage to tissue not intended to be ablated or otherwise treated; (viii) increase in likelihood of success of treatment; and/or (ix) not requiring additional sensors specifically dedicated for contact monitoring. Current approaches use dedicated sensors to measure contact force. Such dedicated sensors increase the cost of the catheter and may affect its ultimate performance. In accordance with several embodiments, the systems described herein use the same sensor (for example, the radiometer) for tissue contact monitoring and for tissue temperature monitoring, thereby decreasing costs and reducing the likelihood of effects on system performance.

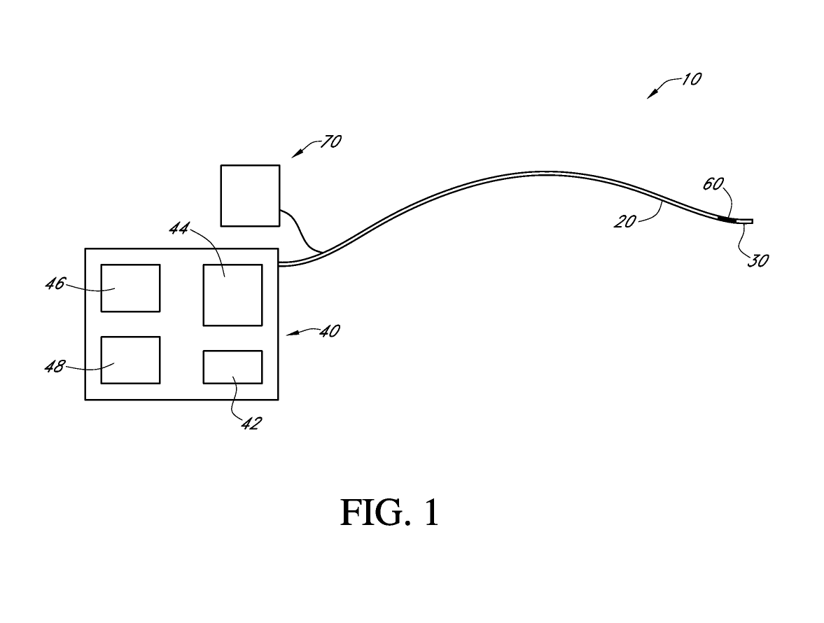

FIG. 1 schematically illustrates one embodiment of an energy delivery system 10 that is configured to selectively ablate or otherwise heat targeted tissue (for example, cardiac tissue, pulmonary vein, other vessels or organs, nerves, etc.). As shown, the system 10 can include a medical instrument 20 comprising one or more energy delivery members 30 (for example, radiofrequency electrodes, ultrasound transducers, microwave antennas) along a distal end of the medical instrument 20. The medical instrument can be sized, shaped and/or otherwise configured to be passed intraluminally (for example, intravascularly) through a subject being treated. In other embodiments, the medical instrument is not positioned intravascularly but is positioned extravascularly via laparoscopic or open surgical procedures. In various embodiments, the medical instrument 20 comprises a catheter, a shaft, a wire, and/or other elongate instrument. A radiometer 60 may be included at the distal end of the medical instrument 20, or along its elongate shaft or in its handle. The term “distal end” does not necessarily mean the distal terminus or distal end. Distal end could mean the distal terminus or a location spaced from the distal terminus but generally at a distal end portion of the medical instrument 20.

In some embodiments, the medical instrument 20 is operatively coupled to one or more devices or components. For example, as depicted in FIG. 1, the medical instrument 20 can be coupled to a delivery module 40 (such as an energy delivery module). According to some arrangements, the energy delivery module 40 includes an energy generation device 42 that is configured to selectively energize and/or otherwise activate the energy delivery member(s) 30 (for example, radiofrequency electrodes) located along the medical instrument 20. In some embodiments, for instance, the energy generation device 42 comprises a radiofrequency generator, an ultrasound energy source, a microwave energy source, a laser/light source, another type of energy source or generator, and the like, and combinations thereof. In other embodiments, energy generation device 42 is substituted with or use in addition to a source of fluid, such a cryogenic fluid or other fluid that modulates temperature. Likewise, the delivery module (for example, delivery module 40), as used herein, can also be a cryogenic device or other device that is configured for thermal modulation. Radiometer 60 may be configured to sense the temperature change of the targeted tissue in response to energy delivery or thermal modulation. The output of the radiometer 60 (for example, the radiometric voltage (Vrad)) may be passed back to the energy delivery module 40.

With continued reference to the schematic of FIG. 1, the energy delivery module 40 can include one or more input/output devices or components 44, such as, for example, a touchscreen device, a screen or other display, a controller (for example, button, knob, switch, dial, etc.), keypad, mouse, joystick, trackpad, microphone or other input device and/or the like. Such devices can permit a physician or other user to enter information into and/or receive information from the system 10. In some embodiments, the output device 44 can include a touchscreen or other display that provides tissue temperature information, contact information, other measurement information and/or other data or indicators that can be useful for regulating a particular treatment procedure.

According to some embodiments, the energy delivery module 40 includes a processor 46 (for example, a processing or control unit) that is configured to regulate one or more aspects of the treatment system 10. The processor 46 may include one or more conventional microprocessors that comprise hardware circuitry configured to read computer-executable instructions and to cause portions of the hardware circuitry to perform operations specifically defined by the circuitry. The output of radiometer 60 is processed by processor 46 so as to detect contact between delivery member 30 and tissue. The module 40 can also comprise a memory unit or other storage device 48 (for example, computer readable medium) that can be used to store operational parameters and/or other data related to the operation of the system 10. The storage device 48 may include random access memory (“RAM”) for temporary storage of information and a read only memory (“ROM”) for permanent storage of information, which may store some or all of the computer-executable instructions prior to being communicated to the processor 46 for execution, and/or a mass storage device, such as a hard drive, diskette, CD-ROM drive, a DVD-ROM drive, or optical media storage device, that may store the computer-executable instructions for relatively long periods of time, including, for example, when the computer system is turned off.

The modules and sub-modules of the system 10 may be connected using a standard based bus system. In different embodiments, the standard based bus system could be Peripheral Component Interconnect (“PCI”), Microchannel, Small Computer System Interface (“SCSI”), Industrial Standard Architecture (“ISA”) and Extended ISA (“EISA”) architectures, for example. In addition, the functionality provided for in the components and modules of computing system may be combined into fewer components and modules or further separated into additional components and modules.

The computing system is generally controlled and coordinated by operating system software, such as Windows 95, Windows 98, Windows NT, Windows 2000, Windows XP, Windows Vista, Windows 7, Windows 8, Unix, Linux, SunOS, Solaris, Maemeo, MeeGo, BlackBerry Tablet OS, Android, webOS, Sugar, Symbian OS, MAC OS X, or iOS or other operating systems. In other embodiments, the computing system may be controlled by a proprietary operating system. Conventional operating systems control and schedule computer processes for execution, perform memory management, provide file system, networking, I/O services, and provide a user interface, such as a graphical user interface (“GUI”), among other things.

The system 10 may also include one or more multimedia devices, such as speakers, video cards, graphics accelerators, and microphones, for example. A skilled artisan would appreciate that, in light of this disclosure, a system including all hardware components, such as the processor 46, I/O device(s) 44, storage device(s) 48 that are necessary to perform the operations illustrated in this application, is within the scope of the disclosure.

In some embodiments, the processor 46 is configured to automatically regulate the delivery of energy from the energy generation device 42 to the energy delivery member 30 of the medical instrument 20 based on one or more operational schemes. For example, as discussed in greater detail in U.S. Patent Application Publication No. 2015/0105765, filed on May 22, 2014, the entirety of which is hereby incorporated by reference herein, energy provided to the energy delivery member 30 (and thus, the amount of heat transferred to or from the targeted tissue) can be regulated based on, among other things, the detected temperature of the tissue being treated.

According to some embodiments, the energy delivery system 10 can include one or more temperature detection devices, such as, for example, reference temperature devices (for example, thermocouples, thermistors, etc.), radiometers and/or the like. Additional details regarding such temperature detection devices are provided in U.S. Patent Application Publication No. 2015/0105765, filed on May 22, 2014, the entirety of which is hereby incorporated by reference herein.

With reference to FIG. 1, the energy delivery system 10 comprises (or is in configured to be placed in fluid communication with) an irrigation fluid system 70. In some embodiments, as schematically illustrated in FIG. 1, such a fluid system 70 is at least partially separate from the energy delivery module 40 and/or other components of the system 10. However, in other embodiments, the irrigation fluid system 70 is incorporated, at least partially, into the energy delivery module 40. The irrigation fluid system 70 can include one or more pumps or other fluid transfer devices that are configured to selectively move fluid through one or more lumens or other passages of the medical instrument 20. Such fluid can be used to selectively cool (for example, transfer heat away from) the energy delivery member 30 during use.

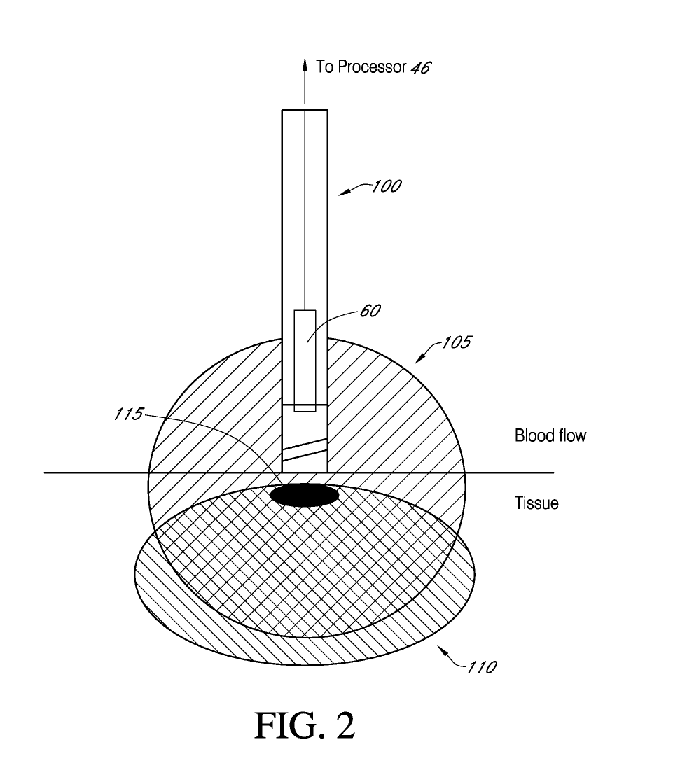

FIG. 2 illustrates an embodiment of a radiofrequency ablation catheter 100 in perpendicular contact with a tissue surface (for example, a cardiac wall or endocardial tissue). The radiofrequency ablation catheter 100 may represent the medical instrument 10 in FIG. 1. A distal tip of the ablation catheter 100 may comprise a member configured to function as both (1) an ablation electrode to deliver energy from the energy source (for example, radiofrequency generator) and (2) an antenna for the microwave radiometer 60. The antenna receives noise power from tissue and transmits the power to the radiometer 60. In some embodiments, the output of the radiometer 60, V_{rad}, is sent to the processor 46 to be processed for tissue contact detection and/or for tissue temperature monitoring.

When in perpendicular contact with the tissue surface, the ablation catheter 100 may have a temperature zone 110 and a microwave sensing zone 105 substantially as shown in FIG. 2. It can be appreciated, however, that clinicians may not always establish perpendicular contact with the tissue surface. If perpendicular contact is not established, the heating zone 110 and the microwave sensing zone 105 may deviate from the positions and shapes shown. In addition, the differing orientations may affect the characteristic impedance of the antenna, thereby resulting in substantially different radiometric responses for the differing antenna/tissue orientations. The different radiometric responses may in turn affect contact detection accuracy and consistency for differing antenna/tissue orientations, and could even result in injury due to operator use deviations or discrepancies. The radiometer may measure temperature of a targeted tissue zone 115 at a depth from the tissue surface.

In some embodiments, microwave radiometers can detect tissue contact based on recognizing differences in properties (for example, dielectric properties) of the surrounding medium (for example, blood vs. heart tissue). For example, dielectric constants may differ such that the characteristic impedance of an antenna, Z_{A}, of the ablation catheter changes as the antenna comes in contact with, or loses contact with, target tissue. In some embodiments, the change in Z_{A} causes a mismatch to the characteristic impedance of the radiometer, Z_{R}. The mismatch can produce an increased amount of microwave reflections at the interface between the antenna and the radiometer. The radiometer detects the increased amount of reflections. In accordance with several embodiments of the methods and systems described herein, the radiometric response that is output to a user, or that is used by the system to determine contact, is designed to be substantially the same, or substantially equivalent or uniform, regardless of antenna/tissue orientation, thereby providing consistency in feedback to the clinicians over various electrode-tissue orientations and over construction variability that may appear during manufacturing.

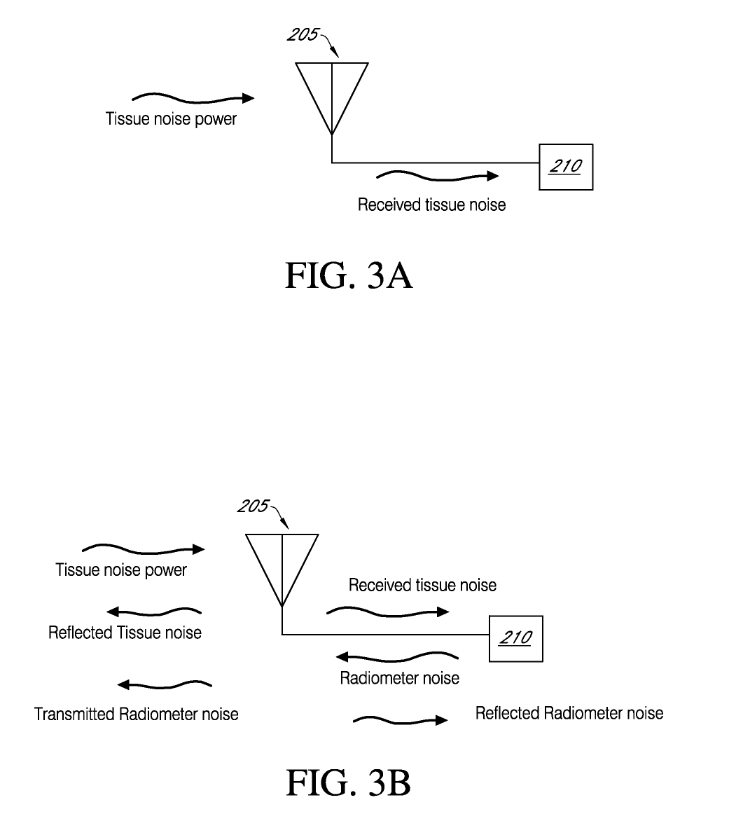

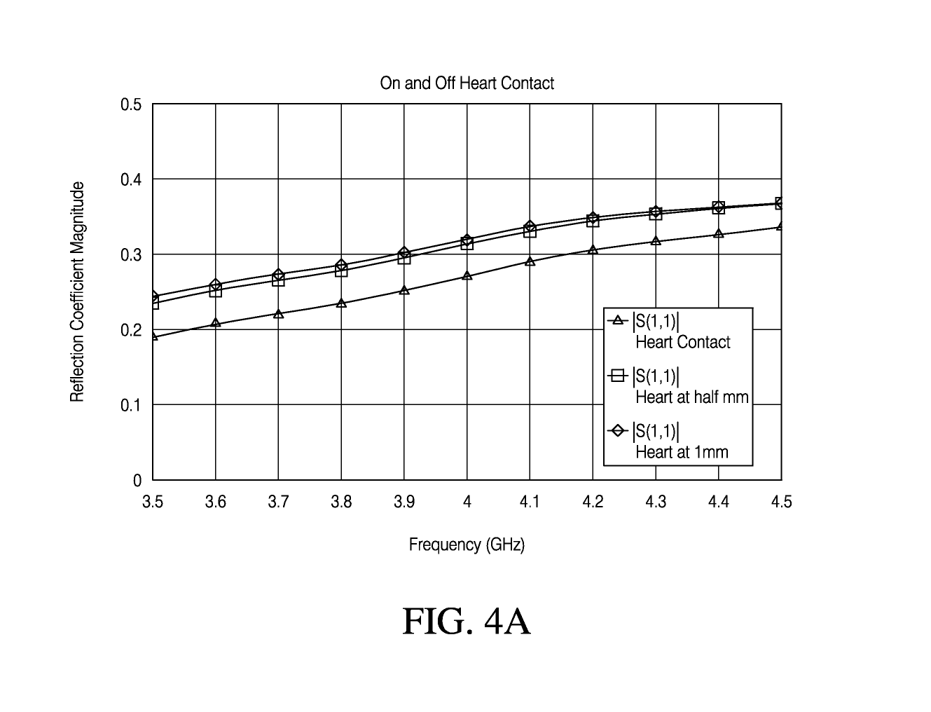

FIG. 3A illustrates one embodiment of operation of an antenna/radiometer system in an ideal setting, where there is no impedance mismatch. In this ideal setting, all of the noise power emitted by the surrounding tissue and received by the antenna 205 and sent to and received by the radiometer 210. However, realistically, impedance mismatches exist between the antenna 205 and the radiometer 210, especially at different regions in the heart (or other organ or vasculature). For example, the impedance mismatches can result in some of the incident power being reflected at the mismatches, as shown in FIG. 3B. In some embodiments, the impedance mismatches result in a measurement environment that is not uniform and that introduces the possibility for measurement errors depending on location (for example, different regions of the heart) and orientation. When some of the power is reflected, the radiometer 210 measures the sum of the incident tissue noise power reduced by the mismatch and the reflected noise power that is emitted by the radiometer 210.

In accordance with several embodiments, error terms caused by the impedance mismatch can advantageously be used to facilitate more uniform detection of contact regardless of orientation. In some embodiments, the impedance mismatch can be characterized by the reflection coefficient:

$$Γ = reflection coefficient = \frac{reflected RF voltage}{incident RF voltage} = \frac{{Z}_{A} - {Z}_{R}}{{Z}_{A} + {Z}_{R}}$$

The radiometer 210 can detect the amount of reflected power and convert that to a temperature change (for example, using the following equation: Noise Power=kTb, where k is Boltzmann's constant, T is temperature in Kelvin and b is bandwidth in Hz.). In some embodiments, the radiometer temperature reading, in terms of the reflection coefficient, can be represented by the following equation:

Tₒᵤₜₚᵤₜ=Tₜᵢₛₛᵤₑ*(1−|┌|²)+T_{radiometer}*|┌|²,

where T_{radiometer} is the equivalent noise temperature emitted from the radiometer input. Before energy is delivered by the ablation catheter, the tissue temperature and the radiometer temperature are constant. Accordingly, in some embodiments, the change in the output temperature is affected primarily by changes in the reflection coefficient. When no energy is being delivered, no actual temperature change occurs; instead, the temperature change is an apparent temperature change. In some embodiments, because the characteristic impedance of the antenna changes, and therefore ┌ changes, when the antenna comes in contact with the tissue, the apparent temperature, Tₒᵤₜₚᵤₜ, also changes when the antenna comes in contact with the tissue.

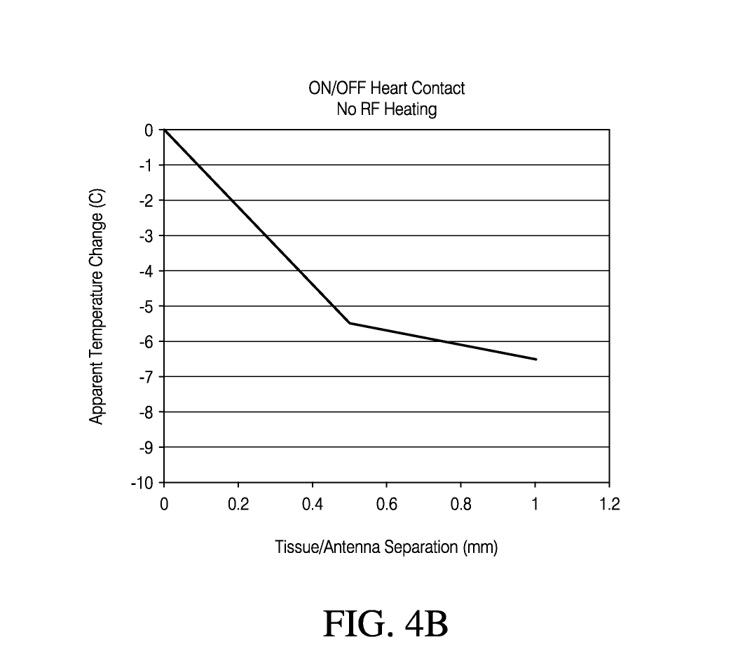

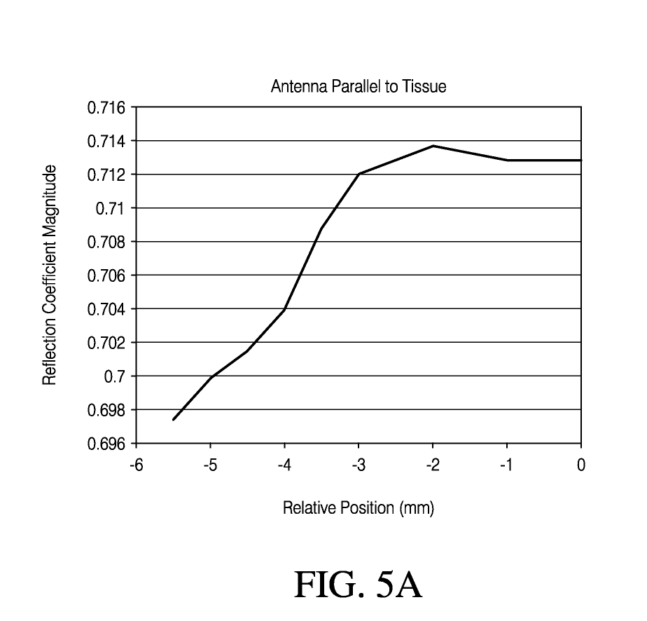

With reference to FIG. 4A, a graph is provided that illustrates the change in magnitude of the reflection coefficient between when the antenna is in contact with heart tissue and when the antenna is not in contact with heart tissue, according to one embodiment. The graph illustrates the reflection coefficient magnitude across various frequencies at contact, at a 0.5 mm separation distance, and at a 1 mm separation distance. As can be seen, the reflection coefficient magnitude is significantly lower when the antenna is in contact with the tissue across the various frequencies. The reflection coefficient magnitude can increase as the antenna is pulled away from heart contact. In accordance with several embodiments, it may be desirable to make the gap between the line at the time of contact and the lines when not in contact to be as large as possible. One embodiment of the resulting apparent temperature change at various distances is shown in FIG. 4B. The abrupt temperature change can be used as an indication, before any heating is applied, that the antenna has contacted or separated from the target tissue (for example, cardiac tissue). In accordance with several embodiments, if the amount of reflection goes down when the antenna touches the tissue surface, it looks like a temperature increase when contact is achieved.

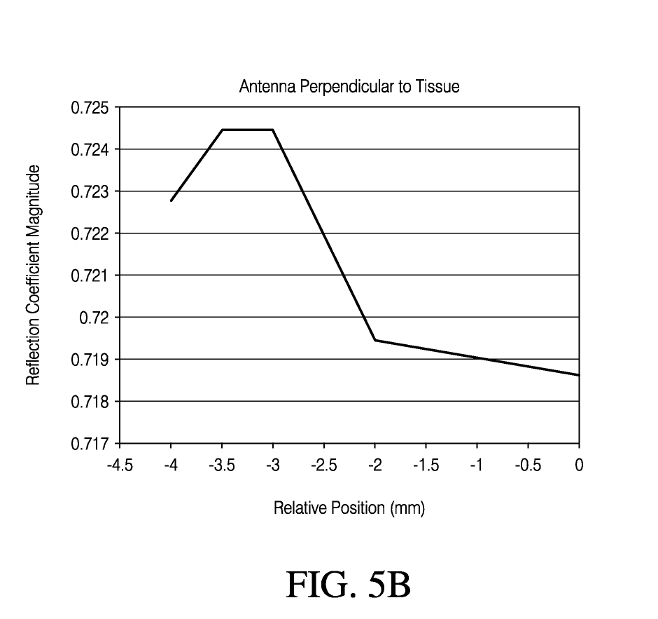

As described above, the radiometric response can vary substantially depending on orientation between the antenna and the tissue surface. FIGS. 5A and 5B illustrate an example of how the radiometric responses may vary between a parallel orientation (FIG. 5A) and a perpendicular orientation (FIG. 5B). In some extreme instances, a perpendicular orientation may result in a temperature increase, and a parallel orientation may result in a temperature decrease because of the way the various impedances are interacting with each other.

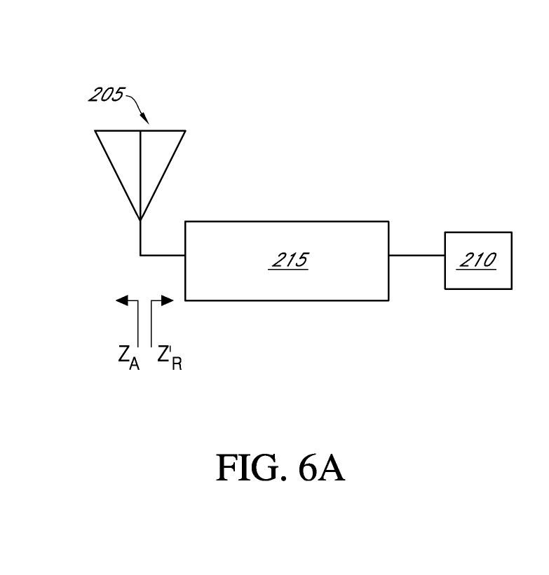

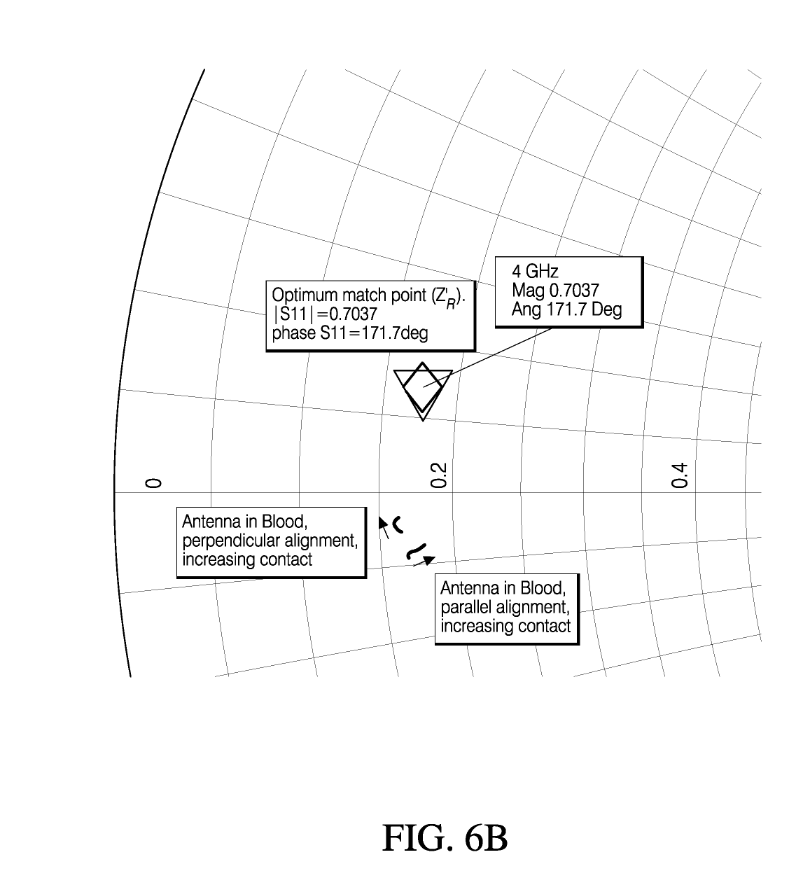

With reference to FIG. 6A, in order to facilitate a substantially similar or equal radiometric response that is not dependent on antenna/tissue orientation, an impedance transformation, or matching, network 215 may be inserted between the antenna 205 and the radiometer 210 to help resolve the mismatches and facilitate substantially similar radiometric responses regardless of orientation of the distal tip, or antenna, with respect to the tissue surface. The impedance transformation network 215 may be used to create a better match to the target tissue to be contacted (for example, heart tissue) compared to other tissue (for example, blood).

In accordance with several embodiments, the impedance transformation network 215 is advantageously tuned to present a desired impedance match to the antenna 205, in accordance with several embodiments. In other words, according to some embodiments, the impedance transformation network 215 can be used to make the reflection from the perspective of the radiometer 210 looking toward the antenna 205 be substantially the same regardless of orientation. In this case, the reflection coefficient observed between the antenna and impedance transformation network is:

$$Γ = reflection coefficient = \frac{{Z}_{A} - {Z}_{R}^{′}}{{Z}_{A} + {Z}_{R}^{′}}$$

where Z′_{R} is the impedance looking from the antenna back into the matching network 215 and radiometer 210.

According to some embodiments, the changes in the antenna impedance Z_{A} (and consequently, the reflection coefficient values), observed in both a perpendicular and parallel orientation can be used to adjust or tune the impedance of the impedance transformation network 215 connected to the radiometer 210 (represented by the impedance Z′_{R} in the above equation) to provide a consistent radiometric response that is substantially equivalent or uniform regardless of orientation. In accordance with several embodiments, the optimum impedance Z′_{R} may be determined from the trajectory of the antenna impedance Z_{A} as contact is made and using Smith Charts and/or other techniques, depending on the operational frequency and other parameters. For example, the impedance at the antenna, Z_{A} (and corresponding S-parameter values of the antenna), can be measured (at a particular operational frequency of the radiometer) as the antenna comes in contact with a tissue surface in both a parallel and a perpendicular orientation. As one example, for an operational frequency of 4 GHZ, the complex conjugate of the one port S-parameters (S11) of the antenna may vary as shown in the portion of the Smith Chart illustrated in FIG. 6B as contact is made. For such an antenna impedance trajectory, an optimum match point for the impedance Z′_{R} may be represented by an S11 (or reflection coefficient) with magnitude and phase values of 0.7037 and 171.7 degrees, respectively.

In accordance with several embodiments, the impedance transformation network 215 is designed such that the contact detection sensitivity for a parallel orientation or a perpendicular orientation is not as optimal as it could be if optimized for only one of the individual orientations. Instead, in some embodiments, the impedance transformation network 215 provides adequate sensitivity for each of the two extreme orientations while still providing accurate contact detection functionality. In some embodiments, the magnitude and/or phase impedance matching values may be within 20% (for example, within 20%, within 15%, within 10%, within 9%, within 8%, within 7%, within 6%, within 5%, within 4%, within 3%, within 2%, within 1%) of the optimum, individual matching values for the separate parallel and perpendicular orientations. In some embodiments, the impedance transformation network 215 is designed utilizing conjugate matching techniques, with a slight amount of mismatch intentionally introduced to allow for orientation-independent contact detection. Once an optimum matching condition is determined, standard matching techniques (such as those described in Pozar 1998, Microwave Engineering 2^{nd} ed., Chapter 5) can be utilized to implement specific lumped and/or distributed elements to achieve the desired impedance Z′_{R}.

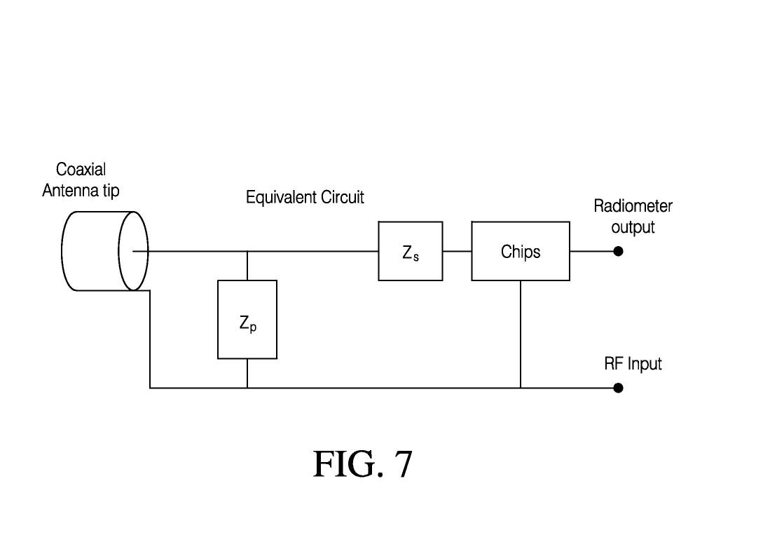

FIG. 7 schematically illustrates an equivalent circuit of the impedance transformation network 215 of FIG. 6A. In various embodiments, elements Z_{P} and/or Z_{S}, which may include filter elements that vary inductance and/or capacitance values (such as LC circuits) or transmission lines (such as thru, open or shorted lines), can be finely tuned to achieve the desired magnitude and phase values determined for the impedance transformation network 215. Inductors and/or capacitors may be inserted between the antenna and the radiometer based on the determined desirable magnitude and phase values for equivalent responses. Properly configured distributed transmission line elements may also be utilized or combined with inductors and/or capacitors to achieve the desired impedance Z′_{R}. For example, embodiments of configurations may include lengths of thru-lines in series or open/shorted lines in parallel.

In some embodiments, the impedance matching transformation network 215 comprises one or more mechanical and/or electrical components. Some of the tuning may be performed by varying components on the radiometer chip(s) and other tuning may be performed by components external to the radiometer chip(s). In some embodiments, components of the antenna, carrier substrate of the radiometer chip(s), and/or irrigation tube adjacent or in contact with the carrier substrate may be modified or adjusted as part of the impedance transformation network 215 (for example, lengths, diameters, materials, antenna helix parameters, etc.). In some embodiments, the irrigation tube is integrated into the matching network 215 by utilizing the tube and carrier substrate to create a quarter-wavelength shorted transmission line. In some embodiments, an initial tuning is performed by adjusting components external to the radiometer (for example, external capacitor or inductor chips or bond wires of varying length) and fine tuning is performed by adjusting components on the radiometer chip itself (for example, capacitor and/or inductor). In some embodiments, the transformation network 215 is mainly reactive and low-loss to reduce any resistance in series with the antenna. The series resistance may desirably be minimal so that the system does not read the series resistance instead of the antenna impedance.

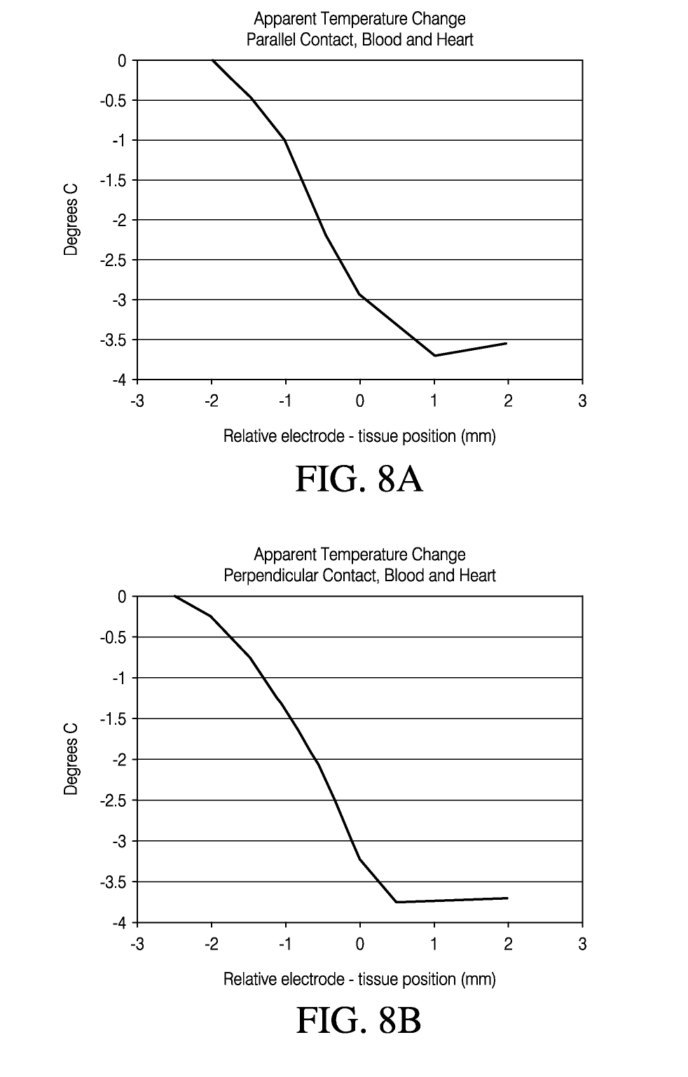

FIGS. 8A and 8B illustrate examples of plots showing uniformity of radiometric response for both parallel (FIG. 8A) and perpendicular (FIG. 8B) contact orientations. The plots illustrate changes in temperature based on electrode/antenna-tissue separation distances. According to some embodiments, the contact detection function shown in the plots of FIGS. 8A and 8B can be approximated by the following equation (for electrode-tissue distances within the range of −2 mm to 2 mm):

$$Δ {T}_{Apparent} = - 2.5 - \frac{6}{π} * arctan ( 3 d ) ,$$

where d is the distance between the antenna/electrode and tissue expressed in mm. The distance between electrode and tissue can be computed as

$$d = - \frac{1}{3} * tan [ \frac{π}{6} * ( Δ {T}_{Apparent} + 2.5 ) ] .$$

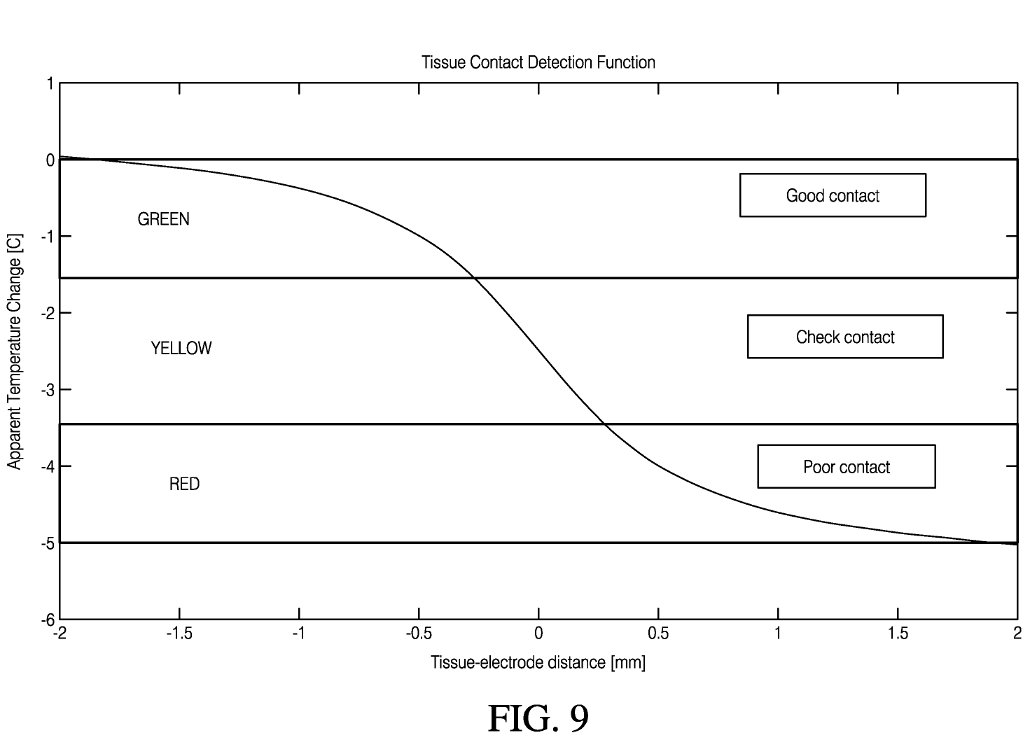

FIG. 9 shows a graph illustrating the dependency between distance d and apparent temperature change, in accordance with an embodiment. FIG. 9 is a graph of a contact detection function and schematically illustrates that a qualitative assessment of contact may be determined for different ranges of measurements and provided to an operator. As shown in FIG. 9, the function or output can be used to determine poor or good contact to tissue, or to alert the operator to check the quality of contact. Alternatively, lookup tables can be used to define the relationship between the electrode-tissue distance and the apparent temperature change. The lookup tables may be associated with processor 46 and can be implemented in hardware and/or software. In some embodiments, quality of contact may be determined, and an output or indication may be provided on a display that is indicative of the level or quality of contact. Different approximations of the relationship between electrode-tissue distance and apparent temperature change may be used in other embodiments. FIG. 9 identifies ranges of apparent temperature change values that may be identified as indicative of poor contact, questionable contact, and good contact. Although not illustrated in color because the figures are represented in black and white, textual labels have been added to indicate colors (red for poor contact, yellow for questionable contact and green for good contact). Alternatively, a quantitative measure of the level of contact between electrode and tissue may be provided by displaying a corresponding bar graph. For example, the bar graph may use at least, but not necessarily limited to, three (for example, three, four, five, six, or more) levels of contact between electrode and tissue. A binary output may be used in some embodiments. In some embodiments, only a qualitative measure is provided and not a quantitative measure.

In some embodiments, the impedance transformation network 215 may be tuned or adjusted such that the apparent temperature increases as tissue contact is made. In other embodiments, the impedance transformation network 215 may be tuned or adjusted such that the apparent temperature drops (rather than increases) as tissue contact is made. This could be achieved using the same techniques previously described above.

In some embodiments, the impedance transformation network 215 may be tuned or adjusted based on, at least in part, surface area contact (for example, percent of electrode coverage) instead of on depth of penetration. Accordingly, the plots in FIGS. 8A and 8B could alternatively show apparent temperature as a function of percentage of total surface area contact. Lesion formation using metal RF electrodes is generally based on the amount of metal-tissue interaction. Accordingly, surface area contact may provide a more accurate indication of quality of contact. In some embodiments, the determination of contact, or the level or sufficiency of contact, may be based on surface area contact or shrouding instead of on depth or separation distance.

The energy delivery system 10 may be configured to operate in two phases or modes: a contact sensing mode and an energy delivery mode. In some embodiments, the system does not enter the energy delivery mode until contact has been determined in the contact sensing mode. The output of the radiometer can be increased or amplified to a higher gain or amplitude during the contact sensing phase. The higher gain or amplification may advantageously result in increased contact detection sensitivity. The amplification may be implemented in hardware (for example, amplifiers, other analog circuit components, etc.) and/or software (for example, digital signal processing). To avoid saturation of the radiometer output signal or addition of excessive noise when energy is delivered to tissue (for example, when higher radiometer output is expected), the gain of the radiometer output may be decreased back to a baseline (where the radiometer may have been calibrated) after contact has been detected and the energy delivery phase is entered. In several embodiments, the contact detection provided herein does not require ablative energy to be delivered.

In some embodiments, the system comprises various features that are present as single features (as opposed to multiple features). For example, in one embodiment, the system includes a single ablation catheter with a single antenna, a single energy delivery radiofrequency electrode and a single microwave radiometer. The antenna and radiofrequency electrode may form a single, unitary, or integral, construct at the distal end of the catheter. A single thermocouple (or other means for measuring temperature) may also be included. The system may comprise an impedance transformation network as described herein. Multiple features or components are provided in alternate embodiments.

In some embodiments, the system comprises one or more of the following: means for tissue modulation (e.g., an ablation or other type of modulation catheter or delivery device), means for generating energy (for example, a generator or other energy delivery module), means for connecting the means for generating energy to the means for tissue modulation (for example, an interface or input/output connector or other coupling member), etc.

Any methods described herein may be embodied in, and partially or fully automated via, software code modules executed by one or more processors or other computing devices. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

In addition, embodiments may be implemented as computer-executable instructions stored in one or more tangible computer storage media. As will be appreciated by a person of ordinary skill in the art, such computer-executable instructions stored in tangible computer storage media define specific functions to be performed by computer hardware such as computer processors. In general, in such an implementation, the computer-executable instructions are loaded into memory accessible by at least one computer processor. The at least one computer processor then executes the instructions, causing computer hardware to perform the specific functions defined by the computer-executable instructions. As will be appreciated by a person of ordinary skill in the art, computer execution of computer-executable instructions is equivalent to the performance of the same functions by electronic hardware that includes hardware circuits that are hardwired to perform the specific functions. As such, while embodiments illustrated herein are typically implemented as some combination of computer hardware and computer-executable instructions, the embodiments illustrated herein could also be implemented as one or more electronic circuits hardwired to perform the specific functions illustrated herein.

The various systems, devices and/or related methods disclosed herein can be used to at least partially ablate and/or otherwise ablate, modulate (for example, ablate or stimulate), heat or otherwise thermally treat one or more portions of a subject's anatomy, including without limitation, cardiac tissue (for example, myocardium, atrial tissue, ventricular tissue, valves, etc.), a bodily lumen (for example, vein, artery, airway, esophagus or other digestive tract lumen, urethra and/or other urinary tract vessels or lumens, other lumens, etc.), sphincters, other organs, tumors and/or other growths, nerve tissue and/or any other portion of the anatomy. The selective ablation, modulation and/or other heating of such anatomical locations can be used to treat one or more diseases or conditions, including, for example, atrial fibrillation, mitral valve regurgitation, other cardiac diseases, asthma, chronic obstructive pulmonary disease (COPD), other pulmonary or respiratory diseases, including benign or cancerous lung nodules, hypertension, heart failure, denervation, renal failure, obesity, diabetes, gastroesophageal reflux disease (GERD), other gastroenterological disorders, other nerve-related disease, tumors or other growths, pain and/or any other disease, condition or ailment.

In any of the embodiments disclosed herein, one or more components, including a processor, computer-readable medium or other memory, controllers (for example, dials, switches, knobs, etc.), displays (for example, temperature displays, timers, etc.) and/or the like are incorporated into and/or coupled with (for example, reversibly or irreversibly) one or more modules of the generator, the irrigation system (for example, irrigant pump, reservoir, etc.) and/or any other portion of an ablation or other modulation system.

Although several embodiments and examples are disclosed herein, the present application extends beyond the specifically disclosed embodiments to other alternative embodiments and/or uses of the embodiments and modifications and equivalents thereof. It is also contemplated that various combinations or subcombinations of the specific features and aspects of the embodiments may be made and still fall within the scope of the disclosure. Accordingly, it should be understood that various features and aspects of the disclosed embodiments can be combined with or substituted for one another in order to form varying modes of the disclosed embodiments. Thus, it is intended that the scope of the disclosure should not be limited by the particular disclosed embodiments described above, but should be determined only by a fair reading of the claims that follow.

While the embodiments disclosed herein are susceptible to various modifications, and alternative forms, specific examples thereof have been shown in the drawings and are herein described in detail. It should be understood, however, that the disclosure is not to be limited to the particular forms or methods disclosed, but, to the contrary, the disclosure covers all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “constructing an impedance transformation network” include “instructing advancing a catheter” or “instructing construction of an impedance transformation network,” respectively. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 mm” includes “10 mm.” Terms or phrases preceded by a term such as “substantially” include the recited term or phrase. For example, “substantially parallel” includes “parallel.”

1.-12. (canceled)

13. A system for facilitating determination of contact of a distal tip of a medical instrument with tissue of a subject comprising:
a connector configured to couple to a medical instrument having an antenna, a radiometer and an impedance transformation network positioned between the antenna and the radiometer, wherein the impedance transformation network is tuned to provide a substantially uniform radiometric response independent of an orientation of the antenna with respect to the tissue as the antenna is brought into contact with the tissue of a subject; and
a processor configured to receive an input signal from the radiometer and provide an output indicative of an amount of tissue contact based upon said input signal.

14. The system of claim 13, wherein the output comprises an apparent temperature change.

15. The system of claim 13, wherein the processor is configured to operate in (i) a contact sensing phase to determine whether an ablation element is in contact with the tissue prior to delivery of ablative energy and (ii) an energy delivery phase upon determination of contact during the contact sensing phase, and wherein a gain of the radiometer output signal is increased during the contact sensing phase.

16. The system of claim 14, wherein contact is determined based upon an abrupt change in the apparent temperature.

17. The system of claim 13, wherein the impedance transformation network is configured to provide a substantially uniform radiometric response for parallel and perpendicular tissue contact orientations.

18. The system of claim 13, further comprising an energy delivery module configured to generate energy to be delivered to the tissue by the medical instrument upon determination of contact.

19. The system of claim 13, further comprising the medical instrument.

20. The system of claim 19, wherein the medical instrument comprises an ablation device including an ablation element positioned at the distal tip of the medical instrument that is configured to deliver ablative energy to the tissue upon determination of contact.

21. The system of claim 13, wherein the tissue comprises cardiac tissue.

22. A medical instrument for facilitating determination of contact of a distal tip of the medical instrument with tissue of a subject comprising:
an antenna;
a radiometer; and
an impedance transformation network positioned between the antenna and the radiometer to provide an output from the radiometer that is not substantially affected by orientation of the antenna with respect to tissue as the antenna is brought into contact with the tissue.

23. The instrument of claim 22, wherein the output comprises an apparent temperature change.

24. The instrument of claim 22, further comprising a processor configured to operate in a contact sensing phase to determine whether the distal tip of the medical instrument is in contact with the tissue prior to delivery of ablative energy and an energy delivery phase upon determination of contact during the contact sensing phase, and wherein a gain of the radiometer output is increased during the contact sensing phase.

25. The instrument of claim 22, wherein contact is determined based upon an abrupt change in the apparent temperature.

26. The instrument of claim 22, wherein the impedance transformation network is configured to provide a substantially uniform radiometric response for parallel and perpendicular tissue contact orientations.

27. The instrument of claim 22, wherein the impedance transformation network is tuned to provide the output from the radiometer that is not substantially affected by orientation of the antenna with respect to tissue as the antenna is brought into contact with the tissue.

28. The instrument of claim 22, in combination with a processor configured to generate an estimate of a distance between the distal tip of the medical instrument and the tissue.

29. The combination of claim 28, wherein the estimate is generated using a function defining an approximate relationship between the distance and a temperature measurement of the radiometric response.

30. The combination of claim 28, wherein the estimate is generated from a look-up table.

31.-35. (canceled)

36. A method of facilitating determination of contact between a medical instrument and tissue that is not dependent on orientation, the method comprising:
determining a first impedance matching value between an antenna and a radiometer of a medical instrument to provide optimum contact sensitivity for perpendicular contact between an energy delivery member of the medical instrument and target tissue;
determining a second impedance matching value between the antenna and the radiometer to provide optimum contact sensitivity for parallel contact between the energy delivery member and the target tissue;
determining a third impedance matching value based on the first and second impedance matching values to provide an adequate level of contact sensitivity whether there is perpendicular contact or parallel contact; and
constructing an impedance matching network to position between the antenna and the radiometer based on the third impedance matching value.

37. The method of claim 36, wherein constructing an impedance matching network based on the determined impedance match comprises determining values of one or more capacitors, inductors or transmission lines of the impedance matching network.

38.-41. (canceled)
